(19) United States
(12) Patent Application Publication
(10) Pub. No.: US 2009/0120439 A1
(43) Pub. Date: May 14, 2009

(54) Method of triggering a ventilator

(76) Inventor: Fred Goebel, Wilhemsfeld (DE)

Correspondence Address: KIMBERLY-CLARK WORLDWIDE, INC.;Catherine E. Wolf, 401 NORTH LAKE STREET, NEENAH, WI, 54956, US

(21) Appl. No.: 11/983,221

(22) Filed: Nov. 8, 2007

(51) Int. Cl. A61M 16/00

(52) U.S. Cl. 128/204.21

(57) ABSTRACT

There is provided a method for controlling breathing gas flow of a ventilator for assisted or controlled ventilation of a patient as a function of a intra-thoracic airway pressure of the patient using a tracheal tube or naso-gastric tube. The intra-thoracic pressure is transmitted to a controller and the information detected is used to control a valve to vent gas from the inhalation tubing of the ventilator, thus triggering an inhalation cycle in the ventilator.

In intensive care therapy, ventilators or respirators are used for mechanical ventilation of the lungs of a patient. The ventilator unit is connected to a hose set; the ventilation tubing or tubing circuit, delivering the ventilation gas to the patient. At the patient end, the ventilation tubing is typically connected to a tracheal ventilation catheter or tube, granting direct and secure access to the lower airways of a patient. Tracheal catheters for critical care ventilation are equipped with an inflated sealing balloon element, or “cuff”, creating a seal between the tracheal wall and tracheal ventilation tube shaft, permitting positive pressure ventilation of the lungs.

State of the art intensive care ventilators enable a medical professional such as a therapist to set, sense or control respiratory parameters such as tidal volume, respiratory rate, respiratory minute volume, flow pattern overtime, time ratio between the inspiration and expiration phase, amplitude of the breathing gas flow, respiratory pressure at the end of an inspiration phase, peak airway pressure, positive end-expiratory pressure (PEEP), as well as volume or flow gradients within the ventilation tubing circuit, thus triggering ventilator generated assist for a spontaneously breathing patient. The diversity of respiratory parameters, in the majority of cases, allows a sufficiently comfortable and safe interaction between ventilator and patient.

In patients with reduced or deteriorated respiratory muscular performance, as can be observed after prolonged periods of tracheal intubation and controlled positive pressure ventilation, the transition from controlled respiratory modes, (wherein the patient is not actively contributing to the exchange of ventilation gas and the ventilation parameters are completely determined by the therapist) to assisted ventilation, (wherein the patient is actively breathing while also receiving tidal support from the ventilator which is sensing and assisting the patients own breathing efforts) can be difficult. This can considerably delay the successful separation of the patient from the intubation tube and the ventilator, also called patient weaning.

A decisive moment in critical care ventilation is the transition from a controlled to an assisted ventilation mode. In modern respiration therapy, patients are kept in an analgo-sedated state, which is sufficiently deep to make the patient tolerate the stimulus of tracheal intubation, but not so intense as to impair the patient's basic neurologic functions such as central respiratory and circulatory regulation. Though the respiratory regulatory functions may be intact, in numerous patients, especially those coming out of a prolonged period of intensive sedation and fully controlled mechanical ventilation, the muscular and mechanical performance of the patent's breathing apparatus can be weakened and deteriorated to such a degree that it is impossible for the patient to release tidal support from the mechanical ventilator and to enter into a sustaining, patient determined, machine assisted breathing rhythm. The mechanism of releasing controlled breathing assist from the ventilator is called triggering.

Modern ventilator types offers two different triggering modes. Tidal support can be released by the patient either by inducing a change in the flow of the ventilation gas inside the patient supplying ventilation tubing (flow triggering), or by lowering the pressure inside that tubing by a certain gradient (pressure triggering). The required flow or pressure change based trigger point is user determined and can be suited to patient's individual triggering capability. Both triggering modes depend on an actual mobilization of ventilation gas volume from the ventilation tube circuit into the patients airways.

Patients whose chest's are mechanically incapable of generating a triggering shift of ventilation gas into the lower airways or are incapable of generating a sufficiently large pressure drop within the patient supplying ventilation tubing, do not receive respiratory support by the ventilator. The performing of chest muscular work (work of breathing) by such patients may be interpreted by the therapist a state of clinical respiratory arrest. The chest muscular and diaphragmatic work by a clinically not-breathing and not-triggering patient may be considerable and, over time, cause fatiguing of the respiratory performance.

Patient breathing activity that is, however, insufficient to release ventilator support, can result from various conditions:

In many cases the chest and diaphragmatic respiratory muscles merely perform isometric contractions not leading to an actual expansion of the lungs and of lung volume. Due to structural (often fibrotic) changes in the lung tissue (an associated stiffening of the lung and a loss of lung tissue compliance) or for example, changes in the composition of the alveolar surfactant, the respiratory apparatus is not able to overcome the initial elasticity of the lungs, which is necessary to open up the various lung compartments, increase their volume and thereby generate the pressure gradient between the distal airways and the patient connected ventilation tubing which is the driving force of external gas exchange. Such isometric or nearly isometric muscle action is usually performed at a high frequency, typically deteriorating in intensity over time, and in many cases leading to the state of total chest mechanical arrest.

In other cases, patients are capable of triggering ventilator support intermittently, yet continue to perform a large number of unproductive isometric breathing actions in between the respirator supported breaths, which are not sensed by the sensor components and remain unnoticed by the ventilator as actual patient breathing activity.

In further cases, conventional ventilator assist may fail or take place only intermittently because of the flow resistance caused by the patient connected ventilation circuit and/or patient intubated tubing itself. Tracheal tubes with a low internal diameter can be particularly dampening (or slowing down) of the flow and pressure changes generated within the distal airways by a patient, to a degree that an actual shift of ventilation gas from the ventilation tubing into the lungs and an associated pressure drop may be not sensed by the ventilator.

In all such cases of isometric muscle action without any volume productive lung expansion (or with an insufficient lung expansion) leading to an insufficient pressure gradient between the distal end of the tracheal tube and the location of the flow or pressure sensing element of the ventilator, or in cases of intermittently triggered support (wherein a significant number of isometric or insufficient respiratory attempts is not sensed and responded by the ventilator) the patient may be performing considerable of work of breathing, over time exhausting his chest muscular and diaphragmatic capabilities and eventually resulting in respiratory fatigue and total mechanical arrest.

Patients in the state of increasing or actual respiratory fatigue must be converted back to controlled ventilation patterns intermittently, enabling the exhausted respiratory apparatus of the patient to recover. In some patients, especially in cases with a history of obstructive lung disease, the successful conversion from controlled to consistently supported ventilation can be achieved only after several days of repeated changes back and forth from assisted to controlled modes, and repeated intermittent episodes of respiratory fatiguing.

In order to overcome the inability of the ventilator to sense the actual onset of mechanical breathing and to prevent unassisted, fatiguing, breathing efforts by the patient, individual respirator types have been equipped with special sensing options able to detect the initial mechanical breathing action performed by the patient's inter-costal and diaphragmatic musculature.

One approach is based on the detection of the initial decrease of the pressure inside the patient's chest cavity; the intra-thoracic pressure, marking the actual onset of mechanical breathing. For that purpose, an intra-thoracic sensor has been suggested. The intra-thoracic sensor detects intra-chest pressure changes without significant time delay and is directly connected to a signal converting pressure sensing unit in the ventilator. The clinical standard for the detection of intra-thoracic pressure dynamics is to place a sensor balloon-equipped probe inside the distal third of the esophagus. The sensor balloon is typically partially inflated, taking up the intra-thoracic force from the organ wall on the sensing balloon. Changes of intra-thoracic volume, resulting in changes of intra-thoracic pressure (following the equation V×p=const), can thereby be sensed continuously and nearly without time delay. While the lung volume, due to reduced lung compliance after prolonged ventilation or due to an underlying lung disease may increase with a certain time delay, other intra-thoracic organs such as the esophagus and the trachea usually communicate pressure changes to a sensor located inside the organ inside the chest, nearly synchronously via the esophageal or tracheal organ wall.

Repeated efforts have been made to provide thoracic triggered assisted ventilation to the therapist. As for example described by Barnard (Esophageal-directed pressure support ventilation in normal volunteers; Barnard et al.; Chest 1999; 115; 482-489) in which a slightly pressured esophageal balloon was connected simultaneously to the inspiratory and expiratory pressure sensor of a Siemens Servo 900 C ventilator. By using the pressure based triggering function of the ventilator (pressure trigger), the modified respirator was able to deliver assist on the basis of intra-thoracic, instead of intra-ventilation tubing pressure, and able to nearly eliminate delays in ventilator assist. The concept of ventilator integrated esophageal/thoracic pressure directed triggering has been proposed by various authors over the past decades but has remained beyond commercial reach.

Another technical approach to sense the actual onset of patient breathing has been to detect electrical currents caused by the diaphragmatic musculature. For that purpose several electrodes are placed on the outside of the abdomen or inside the esophagus on the height of the diaphragm. The currents are amplified by an EMG comparable amplifier, filtered and processed by appropriate software, enabling one to sense the beginning of muscle action, as well as to monitor the muscular performance of a patient (see for example US U.S. Pat. No. 6,584,347). Yet such EMG based interfaces with the patient are expensive, require complex programming and have not been integrated into ventilators.

Previous approaches to reduce triggering work performed by the patient have also involved the measuring of the central airway pressure via an additional pressure measuring tube placed in the distal trachea or integrated in the tracheal tube shaft. The small bore pressure measuring channels, however, rapidly plug up with secretions and not clinically reliable. Other similar approaches teach a tracheal ventilation tube which has a pressure sensor located near the distal end of the tube shaft. The pressure sensor is connected to an electronic signal processor and the signal obtained is used to control various functions in the ventilator. The general concept of distal/tracheal pressure directed triggering has been described in literature repeatedly (as e.g. in Tracheal pressure ventilator control; Banner M J. Blanch P B; Semin Respir Crit Care Med. 2000; 21(3): 233-43). The method is technologically complex, requires specially designed tracheal tubes and has to be suited to the individual ventilator type. Furthermore, respirator triggering on the basis of central airway pressure changes is not capable of sensing the onset of merely isometric breathing work, not resulting in any or only a small shift of ventilation volume.

Another approach to the sensing of the onset of chest wall activity have been motion detecting sensors, placed on the outside of the thoracic and/or abdominal wall (Patient-triggered ventilation: A comparison of tidal volume and chestwall and abdominal motion as trigger signals; Werner Nikischin, Tilo Gerhardt, Ruth Everett, Alvaro Gonzalez, Helmut Hummler, Eduardo Bancalari; Pediatric Pulmonology 1998; 22 (1):28-34). The technology has been shown to be very receptive to artefacts and is therefore difficult to operate in clinical routine.

Triggering on the basis of thoracic impedance changes is another recently described option to reduce patient imposed triggering work. The signal is sensed by a cardiorespiratory monitor, detecting the changes in transthoracic impedance that are associated with inspiration and expiration caused by fluctuations in the ratio of air to fluid in the thorax (Patient triggered synchronized assisted ventilation of newborns. Report of a preliminary study and three years experience; Visveshwara N, Freeman B, Peck M, Caliwag W, Shook S, Rajani K B; J Perinatol 1991; 11(4):347-54). Unfortunately, the impendance based signal can be easily disrupted by cardiac artefacts, lead placement, or change in body position.

There remains a need for a method of trigering a ventilator to reduce and control the amount of breathing work (work of breathing or WOB) performed by a patient being ventilated in a mechanically assisted ventilation mode. There remains a need for a device to enable a therapist to control and gradually increase the work of breathing performed by the patient in order to train and gradually improve the chest mechanical performance of the patient.

### SUMMARY OF THE INVENTION

There is provided a technique of intra-thoracic pressure oriented triggering by a ventilator-type independent, stand alone and simple to operate unit, which is designed to be universally compatible with either flow or pressure based ventilator triggering modes, whereby the unit operates fully ventilator-independent, i.e., not requiring any electrical connection with the ventilator or modification of ventilator software or hardware.

Subsequent to sensing the initial decrease in intra-chest pressure, marking the onset of patient breathing activity, a pressure release valve which is inserted into the ventilation tubing circuit is opened, thus initiating a pressure drop or flow change inside the ventilation tubing, whereby the generated pressure or flow gradient is sufficiently large to be sensed by the ventilator integrated pressure or flow sensors. The associated work of breathing which is performed by the patient can thus be minimized and to a large degree controlled by the therapist. Respiratory fatigue of the patient in the transition phase from controlled to assisted patient ventilation can be reduced or prevented and patient weaning accelerated.

### BRIEF DESCRIPTION OF THE DRAWINGS

FIG. 1 describes the basic set up of the inventive device and its integrated individual components.

FIG. 2 describes the individual functional components inside the main unit.

FIG. 3 shows alternative embodiments of pressure release valves.

FIG. 4 shows the timely relation between intra-chest pressure, distal and proximal airway pressure, intra-balloon pressure, patient breathing and ventilator assist.

### DETAILED DESCRIPTION OF THE INVENTION

The following describes a technology/device developed to accelerate and better control the transition from controlled to assisted (or supported) ventilation modes. By triggering ventilator support on the basis of the detection of relative changes in the intra-chest pressure (intra-thoracic pressure) of a patient, patients can be converted to assisted breathing significantly earlier, and in a manner not requiring any direct communication between the inventive device and the ventilator. As such, the invention represents a relatively simple, easy to apply, universally compatible device for more efficient patient weaning.

The beginning of a breathing cycle can be detected either by a chest volume expansion associated pressure change inside the cuff of a tracheal or tracheostomy tube. Alternatively, an intra-thoracic pressure change can be detected by a pressure sensing element located in the esophageal section of a naso-gastric tube (NG tube).

Tracheal or tracheostomy tubes carry an inflatable cuff at their distal end, sealing the trachea and permitting active, machine operated ventilation of the lungs with positive inflation pressures. The cuff filling is controlled via a shaft integrated inflation channel and a shaft connected piece of inflation tubing. The inflation line is usually equipped with a check valve. Due to the intra-chest position of the cuff, the cuff pressure responds to force changes transmitted via the tracheal wall onto the inflated tube cuff. Intra-thoracic force changes are transmitted via the cuff inflation line and can be detected continuously by a pressure transducing module.

Changes of pressure within the cuff of an intubated, spontaneously breathing patient have been found to be predominantly influenced by the intra-thoracic force, resting on the tracheal inflated cuff (Badenhorst C. H.; Changes in tracheal cuff pressure during respiratory support; Critical Care Medicine 1987; 15:300-302). To a significantly lesser degree and time delayed, the cuff pressure is influenced by changes in distal airway pressure, which effect the cuff pressure via the distal, lungwards directed face of the intubated cuff being exposed to the tracheo-bronchial airway. Tracheal tube cuffs, as outlined in U.S. Pat. Nos. 6,526,977 and 6,802,317, incorporated herein in their entirety for all purposes by reference, have shown to be extremely rapid in response to changes in intra-thoracic and distal airway pressure.

Alternatively, intra-thoracic pressure changes may be sensed by an esophageal sensor element positioned in the esophageal section of an esophageal inserted probe. Such a probe can be a balloon equipped naso-gastric tube used to decompress the abdomen of a ventilated patient or for delivery of feeding solutions into the patient's stomach. In critical care therapy, gastric (enteral) feeding is usually performed via naso-gastric decompression catheters (NG-tubes), which are primarily used to release pressure building up in the stomach of a patient. Excessive gastric pressure may result from the accumulation of liquid intestinal secretions, feeding solution applied into the stomach or duodenum, abdominal motility, body movement or positioning of the patient, or through normal formation of gas. For decompression of gastric pressure and drainage of gastric contents, such patients may be intubated with naso-gastric or oro-gastric tubes or probes. An example of one such stomach probe is described in German Utility Model Application No. 202006002832.3. Another is described in U.S. Pat. No. 6,551,272 B2, which is hereby incorporated herein in its entirety for all purposes by this reference.

The above mentioned applications describe tracheal ventilation tubes and gastric probes integrating membrane-like balloon elements, giving the most sensitive, timely, accurate and continuous reflection of intra-thoracic force resting on the balloon through the organ wall.

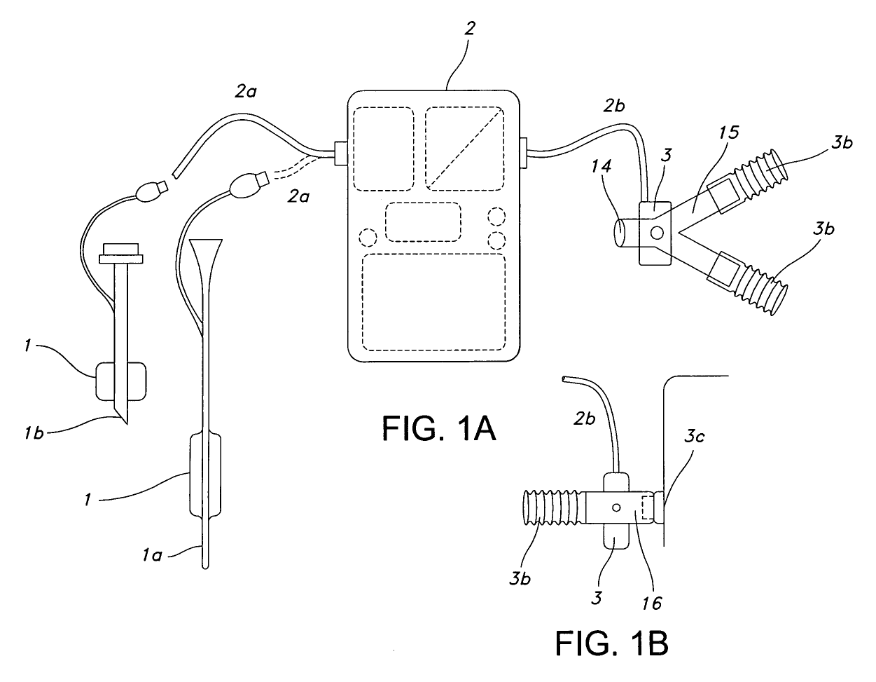

As shown in FIG. 1, a pressure sensing balloon 1 on either a naso-gastric tube 1a or a tracheal tube 1b, which is continuously reading relative force changes (i.e. pressure) within the thoracic cavity of a patient, communicates with the controller or main unit 2. This communication may take place via a pneumatic tube or electrical cable connection 2a or other means (e.g. wirelessly). The main unit 2 includes a means for receiving and interpreting the incoming data from the balloon 1 and for sending a signal to and controlling the pressure release unit 3. The interpreting and controlling means may include a pressure transducing module or a signal amplifying input module, a pneumatic pump mechanism, and/or a logic control unit. Thus the main unit 2 integrates parameter input and signal reading options.

The main unit 2 communicates with the pressure release unit 3 via a pneumatic tube or electric cable 2b or otherwise (e.g. wirelessly). The pressure release unit 3 may include a Y-shaped union piece 15 joining the patient ends of the inspiratory and expiratory of the ventilation tubing 3b and the patient proximal portion of the tracheal ventilation tube 14. It may alternatively be integrated into a piece of tubing 16, which is inserted at any position within the ventilation tubing, preferably between the ventilation tubing connector of the ventilator 3c and the ventilator tubing 3b (FIG. 1b). In either case, when the pressure release unit 3 receives a signal from the main unit 2, a valve 18 (not shown) within the pressure release unit 3 opens and releases ventilation gas from the ventilator circuit to the outside atmosphere.

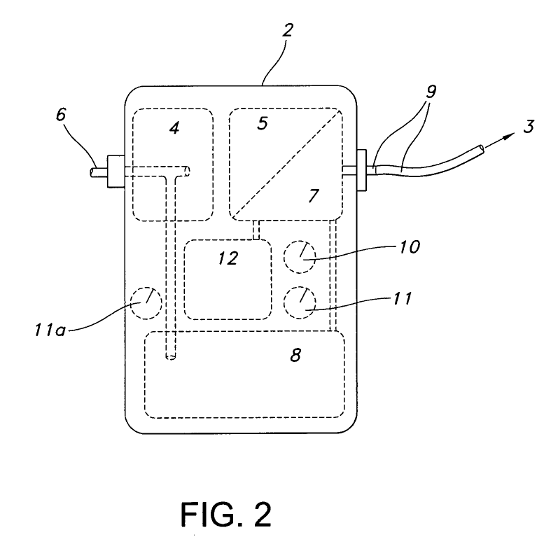

Turning now to FIG. 2; the main unit 2 may contain a pressure transducing module 4 and an analog-digital (A/D) converting module 5 converting the analog pressure signal into digitalized data. The transducing module 4 may be equipped with a communication port 6 for connection with a balloon 1 based sensor tube 1a or 1b, or an electric cable, in case an electronic pressure sensor is used on the pressure sensing catheter. Connected to the A/D converting module 5 may be a processor based control unit 7. The control unit 7 may control an electromagnetically operated pump mechanism 8 which may intermittently regulate the filling of the sensor balloon 1 to a user-determined value. The control unit 7 may be operatively connected with the pressure release unit 3 which may include a preferably electromagnetically operated valve mechanism. The control unit 7 may include a manual setting option 10 for the sensor balloon 1 filling pressure. The control unit 7 may further include a manually entered option 11 for a user-determined response delay period to allow for setting a time interval between the detection of respiratory onset and pressure release through the pressure release unit 3.

Unit 7 may further include a manual setting option to adjust the length of the pressure release period 11a to the response properties of the individual ventilator.

The control unit 7 may also be connected to a LCD display module 12 which may (continuously) display the sensor balloon 1 filling pressure. This may also insert indication markings, indicating the onset of patient breathing and the following onset of mechanical tidal support, thus enabling the user to visually confirm the timely relationship between patient breathing and ventilator onset.

The unit may optionally be equipped in accordance to the work of breathing (WOB) monitoring function as described in DE 102 13 905 and related U.S. Pat. No. 7,040,321. The WOB option (continuous display of ventilated tidal volume over intra-thoracic sensed pressure) depends on the availability of an additional parameter; the tidal volume being moved in and out of the patients airways. The parameter should be sensed continuously, e.g. by a flow detecting sensor inserted into the ventilation tubing circuit. The main unit 2 may be adjusted accordingly in order to display the reiterating, color coded WOB loops, as outlined in the above patents.

While the balloon-based sensors described above are preferred, other means of pressure detection may also be used. The main unit 2, for example, may be designed for pressure sensing by an electronic pressure sensor like the intra-thoracic pressure sensor discussed above, basically eliminating the need for a pressure transducing and pneumatic pump module. Additionally, instead of pressure gradient based signal analysis, the pressure release unit 3 can be controlled by a signal analyzing autocorrelation algorithm, identifying the onset of patient breathing signal-morphologically, as outlined in DE 102 13 905 and U.S. Pat. No. 7,040,321. The main unit 2 may also be equipped with an entering option for an auto-correlation coefficient (reaching from −1 to +1), chosen by the user, and functioning as a triggering threshold.

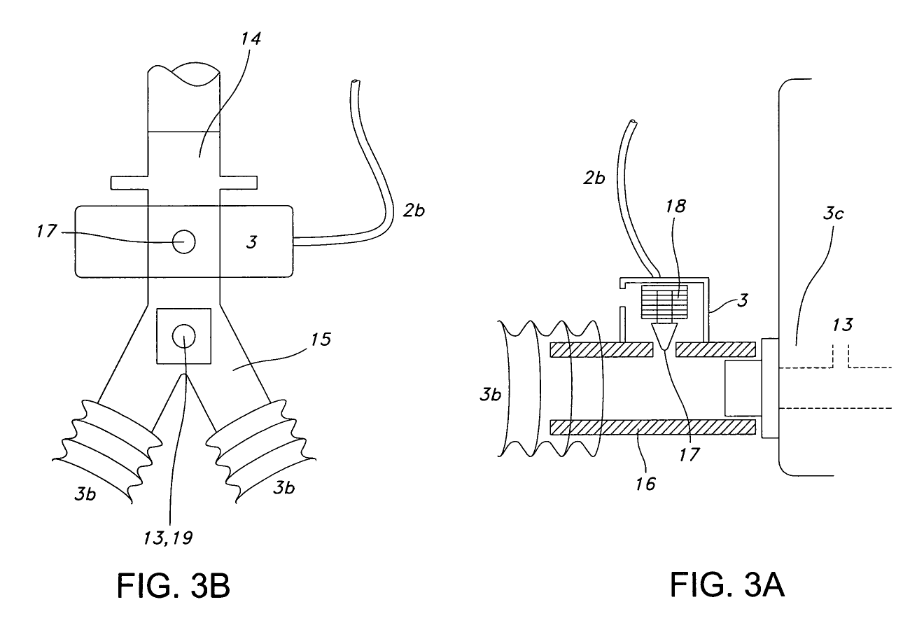

As illustrated in FIG. 3a, the pressure release unit 3 is preferably located as close to the pressure sensing module 13 of the ventilator as possible, in order to prevent dampening effects and to create the least possible triggering delay. In case of ventilator unit integrated pressure sensors 13, the pressure release unit 3 can be placed directly between the unit connector closest to the triggering responsible sensor 13 and the ventilator tubing 3b (FIG. 3a), adjacent the ventilator. The pressure release unit 3 would most basically be designed as an inserted tube piece 16 which is sized to fit the tubing connections and adaptors within the ventilation tubing circuit, which in nearly all cases complies with specific industrial standards. The specific connector dimensions of the pressure release unit 3 carrying the tube segment 16 makes the inventive device compatible with almost all current types of ventilators, so that no further communication (e.g. electrical connections) between the invented device and the ventilator is required. The tube piece 16 may integrate an opening 17 and, for example, an electro-mechanically opening and closing element or valve 18.

In case the ventilator pressure sensor 13 is located adjacent to the patient proximal portion of the tracheal ventilation tube 14 as illustrated in FIG. 3b, the release pressure function should be inserted into the tubing circuit where the expiratory and inspiratory limbs 3b of the circuit meet, adjacent to the patient. This may be done using a modified Y-piece union 15. The Y-piece 15 also includes an opening 17 and, for example, an electro-mechanically opening and closing element or valve 18 (not shown).

Closing element or valve 18 releases pressure from the ventilation tubing circuit over a software defaulted period of time, being sufficient to trigger a supporting tidal action from the majority of ventilator types. During this period valve 18 preferably is in an activated, powered state, opening the ventilation tubing to the ambient environment through opening 17. In order to match the release period with the specific response properties, determined by the individual interaction of chest mechanics, ventilation tubing and ventilator, an option for manual tuning of the pressure release period may be included in the device. In the non-activated state, valve 18 moves into a close-position, securely locking opening 17.

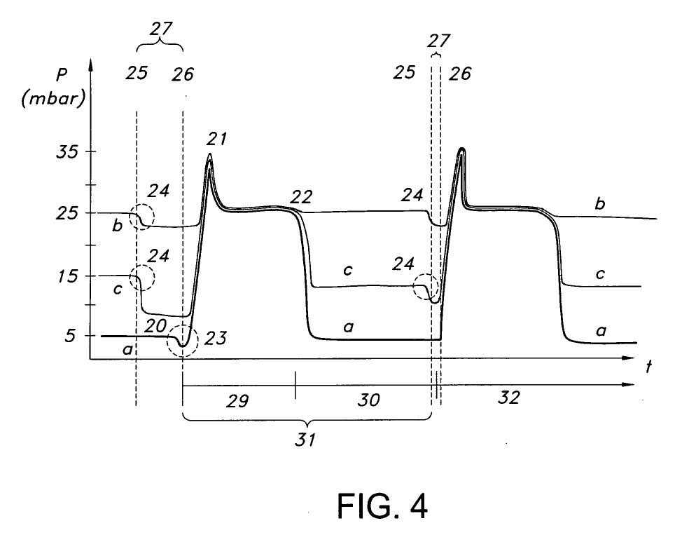

Curve a of FIG. 4 shows the respiratory pressure in the patient airway as it can be sensed inside the ventilation tubing connected to the patient. In this graph, pressure is in millibars on the vertical (Y) axis and time is on the horizontal (X) axis. In ventilated patients, in the phase between flow of tidal volume in or out of the patient, the pressures inside the ventilation tubing and within the lower patient airways equalize. In ventilation therapy the achieved resting pressure is usually kept on a low positive level, in most cases between 5 and 10 mbar, the PEEP pressure 20 (positive end-expiratory pressure), with the intention of keeping the lung compartments at least partially open and to prevent a collapse of the distal, gas exchanging portions.

Curve a also shows the inspiratory 29 and expiratory 30 portion of a respiratory 31 or breathing cycle, as it is interpreted by a conventional ventilator. Inspiration typically shows a steep initial pressure increase to a peak pressure value (PEAK) 21, from where the pressure falls back to an elevated inspiratory pressure plateau (PLATEAU) 22 (resulting from the subsequent expansion of lung volume and opening of the various lung compartments). At the end of the inspiratory plateau the expiratory phase begins. The airway pressure decreases, returns to PEEP 20 level and remains on PEEP 20 level till the next inspiratory phase begins. Triggering of tidal assist has to be performed within this so called post-end-expiratory phase.

The beginning of a machine assisted inspiratory phase is usually marked by an initial respiratory pressure drop (IRPD) 23 in the respiratory pressure curve. The pressure drop (or the resulting flow change in the tubing circuit) is sensed by the ventilator or ventilation tubing integrated pressure sensor equipment. If a certain pressure reduction, which has been set by the user as a triggering threshold, has been reached, the ventilator releases the tidal support to the patient initiated breath.

Curve b of FIG. 4 shows the intra-cuff pressure inside a tracheal ventilation tube cuff. As discussed above, the cuff filling pressure reflects an intra-thoracic pressure decrease when patient breathing is initiated and so can be used to detect the onset of patient breathing (OPB) 24 activity.

Curve c of FIG. 4 displays the filling pressure within an inflated intra-esophageal balloon, which is fully exposed to breathing associated intra-chest pressure changes, therefore also being capable of detecting the onset of patient breathing (OPB) 24.

In conventionally triggered and ventilated patients without the inventive device, as shown in the first respiratory cycle 31 of FIG. 4 in which the patient is triggering ventilator assist on the basis of an initial respiratory pressure drop (IRPD) 23 in the ventilation tubing (sensed by the ventilator), the onset of breathing muscular action (BMO) 25 can appear considerably earlier than the tidal assist 26, which is delivered by the ventilator. In the intermediate period the patient performs unassisted, potentially fatiguing work of breathing (WOB) 27. This time-delay in the receipt of breathing assistance should be avoided.

At the start of the second respiratory cycle 32 using the inventive device, the intra-thoracic pressure decrease, marking the onset of patient breathing 24, is sensed by the inventive device which releases volume from the patient supplying ventilation tubing via the valve 18 (FIG. 3a), thereby generating the required flow or pressure change in the tubing, which in turn triggers the ventilator's tidal support. The main unit senses the onset of ventilator support by an increase in esophageal/tracheal cuff pressure. Once the increase in esophageal/tracheal cuff pressure is sensed, the triggering window and the valve 18 are closed until the pressure returns to the default pressure and optionally stays there for a certain defined period; within that period the slope of delta pressure/delta time should be neutral or negative. The window does not open if a positive slope appears. As can be seen in FIG. 4, at the start of the second respiratory cycle 32 using the inventive device in intra-thoracic triggering, the time interval between the onset of breathing muscular activity (BMO) 25 and the tidal assist by the ventilator (TA) 26 can be considerably reduced. As a result the work of breathing 27 is also substantially reduced.

By defining the response delay interval between the moment of pressure sensing and the moment of the release of gas from the ventilator tubing, which can be manually set by the therapist, the amount of patient performed WOB 27 during a respiratory cycle can be minimized, thus enabling an early, successful and stable transition from a controlled to a supported ventilation mode. Alternatively, by a gradual increase of the response delay interval, the patient performed amount of WOB 27 can be manipulated to produce accelerated chest mechanical training and weaning of the patient from the ventilator. The response delay interval can be entered by a manual input function.

The thoracic pressure gradient (delta P), which defines the trigger sensitivity of the invented device, can be defined as a simple gradient value. Alternatively, the trigger threshold, can be user defined as a gradient over time (delta P over delta t), whereby the slope of the underlying differential is preferably high for low pressure gradients, and low for larger gradients.

In order to create the best possible synchronicity between patient breathing activity and ventilator assist, the unit control software may make the release of the triggering impulse dependent upon the fulfillment of certain criteria, for example;
triggering may not be possible if the sensed intra-thoracic pressure shows an increase (positive slope of pressure curve)
triggering may only be released within a (narrow) defined range of thoracic pressure, whereby the defined pressure range should be equal to or close to the user defined filling pressure of the sensing balloon inside the thoracic cavity
triggering within that defined pressure range may depend on a certain period of signal stability within that range (neutral or negative slope of pressure curve over a certain period of time) in order to prevent e.g. unintended triggering during the phase the thoracic pressure curve is returning to its base after a supported breath. After that “pressure stable” episode, the “triggering window” opens.
the device may detect and indicate/display tidal support by the ventilator by an increase of intra-thoracic pressure or a certain pressure differential (slope) to be reached.
alternatively, within such a defined triggering range and time window, triggering may be released on the basis of curve morphology and an analyzing underlying auto-correlation algorithm.

The monitoring of the patient's chest mechanical performance as repeating work of breathing (WOB) loops requires, next to a continuous measurement of thoracic pressure, the additional measurement of the volume of ventilation gas which is moved in and out of the patient. The shifted volume can be sensed by a flow measuring sensor element 19, which may be integrated into the pressure release unit 3.

The cuff or balloon for the tracheal tube/tracheostomy cannula, or the balloon for the gastric probe, is preferably made from a stretchable thin plastic film with a wall thickness of less than 0.02 mm, and in particular a wall thickness in the range from 0.01 to 0.005 mm. The cuff or balloon can be subjected to a fill pressure of 25 mbar, and preferably to a fill pressure in the range between 10 and 20 mbar. The plastic film may comprise a thermoplastic polyurethane elastomer, and it should have a tension modulus of at least 10 MPa at 300 percent expansion in accordance with ASTM D 412.

The microthin-walled cuff or balloon 1 of a ventilator tube or gastric probe makes it possible to detect very small intra-thoracic pressure fluctuations via the tracheal or esophageal balloon membrane with high measurement precision and largely without a time delay.

A tracheally placed micro-thin balloon typically can be filled in a pressure range of 20 to 30 mbar in adults and 5 to 15 mbar in small children and infants. An esophageal based balloon may be typically filled in a range of 5 to 30 mbar.

The general principle of releasing assist from a conventional flow or pressure triggered ventilator by inducing a pressure drop within the patient supplying ventilation tubing, as being described in this invention, can also be combined with other signal detecting principles/units, determining the onset of breathing e.g. by electromyography, distal airway pressure changes, motion detecting surface capsulas or thoracic impedance changes.

As will be appreciated by those skilled in the art, changes and variations to the invention are considered to be within the ability of those skilled in the art. Such changes and variations are intended by the inventors to be within the scope of the invention. It is also to be understood that the scope of the present invention is not to be interpreted as limited to the specific embodiments disclosed herein, but only in accordance with the appended claims when read in light of the foregoing disclosure.

What is claimed is:

1. A method of trigering a ventilator having an inhalation circuit for ventilation of a patient, comprising determining an intra-thoracic airway pressure of the patient and controlling a flow of breathing gas from the ventilator by venting the gas from the inhalation circuit of the ventilator as a function of the determined intra-thoracic pressure.

2. The method of claim 1 wherein the intra-thoracic pressure is detected using an endotrachael tube.

3. The method of claim 1 wherein the intra-thoracic pressure is detected using a naso-gastric tube.

4. The method of claim 3, wherein the endotracheal tube has a cuff that made from a stretchable thin plastic film with a wall thickness of less than 0.02 mm.

5. The method of claim 4, wherein the cuff is made from a film of thermoplastic polyurethane elastomer with a modulus of tension of at least 10 MPa at 300 percent expansion in accordance with ASTM D 412.

6. The method of claim 1 wherein said gas is vented to the ambient atmosphere via a valve.

7. A system for trigering a patient ventilator comprising a means for detecting an intra-thoracic pressure of a patient wherein said means are in operable communication with a controller, and a vent valve located in a ventilator tubing line and controlled by said controller.

8. The system of claim 7 wherein said controller comprises a transducing module that receives a pressure reading from a tracheal or naso-gastric tube, a converting module that converts the pressure reading from an analog to a digital signal, and a control unit that receives said signal and commands a vent valve to open in response to said pressure reading.

9. The system of claim 7 wherein said vent valve is located adjacent to a ventilator.

10. The system of claim 7 wherein said vent valve is located adjacent to a patient.

11. The system of claim 10 wherein said vent valve is part of a union piece connecting a patient proximal portion of a ventilator tubing and inspiratory and expiratory ventilator tubing.

12. The system of claim 7 having no electrical connections with the ventilator.

13. The system of claim 7 wherein said controller opens said vent valve in response to a drop in said intra-thoracic pressure.

14. The system of claim 13 wherein said controller may introduce a time delay between receipt of said pressure and opening said vent valve.

15. The system of claim 14 wherein said time delay may be adjusted by a medical professional.

16. The system of claim 7 wherein said means for detecting an intra-thoracic pressure of a patient comprises a tracheal tube balloon or naso-gastric tube balloon.
